(19) United States
(12) Patent Application Publication
(10) Pub. No.: US 2024/0066506 A1
(43) Pub. Date: Feb. 29, 2024

(54) CATALYST FOR HYDROGENATION REACTION OF CARBON DIOXIDE, METHOD OF PREPARING SAME, AND METHOD OF SYNTHESIZING LIQUID HYDROCARBON COMPOUND USING SAME

(71) Applicant: RESEARCH & BUSINESS FOUNDATION SUNGKYUNKWAN UNIVERSITY, Suwon-si (KR)

(72) Inventors: Jae Hoon KIM, Seongnam-si (KR); Won Joong YOON, Suwon-si (KR); Heun Tae JO, Suwon-si (KR); Muhammad Kashif KHAN, Suwon-si (KR)

(73) Assignee: RESEARCH & BUSINESS FOUNDATION SUNGKYUNKWAN UNIVERSITY, Suwon-si (KR)

(21) Appl. No.: 18/458,510

(22) Filed: Aug. 30, 2023

(30) Foreign Application Priority Data
Aug. 31, 2022 (KR) 10-2022-0109781

(51) Int. Cl. B01J 23/78; B01J 23/04; B01J 37/02; B01J 37/03; B01J 37/08; B01J 37/16; C07C 1/12

(52) U.S. Cl. CPC B01J 23/78; B01J 23/04; B01J 37/0236; B01J 37/038; B01J 37/08; B01J 37/16; C07C 1/12; C07C 2521/04; C07C 2523/04; C07C 2523/745

(57) ABSTRACT

Disclosed is a method of preparing sodium-catalyzed iron-aluminum inorganic catalyst for catalyzing a hydrogenation reaction of carbon dioxide. The method of preparing sodium-catalyzed iron-aluminum inorganic catalyst includes: a first step of mixing a precipitant solution formed by dissolving a basic precipitant with a reaction solution formed by dissolving an iron (Fe) precursor material and an aluminum (Al) precursor material, thereby forming a suspension solution; a second step of aging the suspension solution; a third step of separating powder from the aged suspension solution; a fourth step of drying the separated powder and then performing a first heat treatment, thereby forming first catalyst powder; and a fifth step of adding and stirring the first catalyst powder and a sodium precursor material to water and evaporating the water and then performing a second heat treatment, thereby preparing a sodium-introduced iron-aluminum inorganic catalyst.

### BACKGROUND OF THE INVENTION

### 1. Field of the Invention

The present invention relates to a catalyst applicable to synthesize a liquid hydrocarbon compound via a direct reaction between carbon dioxide and hydrogen, a method of preparing the same, and a method of synthesizing a hydrocarbon compound using the same.

### 2. Description of the Related Art

The development of technology of effectively using carbon dioxide (CO₂) to form fuels and chemicals is a highly promising strategy for establishing a CO₂-neutral and sustainable society. A thermocatalytic conversion of carbon dioxide, which is mainly based on the reaction of reverse water-gas shift (RWGS, Reaction Formula 1) and the subsequent reaction of Fischer-Tropsch synthesis (FTS, Reaction Formula 2), is considered as a promising approach for producing flat-form compounds formed by adding values such as gaseous fuels having 1 to 4 carbon atoms (C1-C4), liquid fuels having 5 carbon atoms or more (C5+), olefins, acids, alcohols, and aromatics.

CO₂(g)+H₂(g)→CO(g)+H₂O(g) [Reaction Formula 1]

CO(g)+H₂(g)→CₙHₘ(g) [Reaction Formula 2]

An iron (Fe)-based catalyst and a cobalt (Co)-based catalyst are mainly used as the FTS reaction catalyst for producing alkanes, alkenes, and oxygenates by using synthesis gas as a mixture of hydrogen (H₂) and carbon monoxide (CO). Many studies have been conducted to use these catalysts as catalysts for the hydrogenation reaction of carbon dioxide.

The Co-based catalyst is very effective for the FTS reaction at a relatively low temperature (<240) because of high chain growth probability (0.94 or higher), high circulation rate, high selectivity for linear paraffins, low WGS reaction activation, high inactivation resistance to water molecules formed during FTS, and high long-term stability. When producing long-chain hydrocarbons under typical FTS reaction conditions, the metallic Co center has been considered a major active site for hydrogenation of carbon monoxide. Recently, it has been proposed that the hydrogenation activity of carbon monoxide on Co₂C is possible when producing lower olefins.

However, the direct carbon dioxide hydrogenation reaction on the Co-based catalyst has excellent long-term catalyst safety, however, a main product is methane (CH₄) and the yield of C5+ hydrocarbons (<5% at gas hourly space velocity (GHSV) ≥4000 mL (g⁻¹ h⁻¹) is very low. The high methanation activity of carbon dioxide in Co-based catalysts is due to the absence of active sites capable of catalyzing RWGS and the C/H ratio on an active surface that induces hydrogenation of CO₂ adsorption species lower than the ration of chain extension reaction.

In order to solve the problem of the Co-based catalysts, a method of respectively and directly converting carbon dioxide into light olefins and ethanol by using CoFe and CoNi catalysts, which are two-component metals, has been proposed. However, designing Co-based catalysts still exists as a difficult technical problem, due to the lack of basic and comprehensive understanding for synthesizing long-chain hydrocarbons directly from carbon dioxide.

The Fe-based catalyst shows similar catalyst performance regardless of using CO or CO₂ as a feed. For Fe-based catalysts, an RWGS reaction of CO₂ on an Fe₃O₄ site may be catalyzed through an oxidation-reduction cycle, and then a continuous FTS reaction on an Fe₅C₂ site may produce C5+ hydrocarbons at high yield (at least about 20% or higher at GHSV≥4000 mL g⁻¹ h⁻¹). However, the Fe-based catalyst has a problem that long-term catalyst safety is deteriorated because oxidation is catalyzed due to water produced during the RWGS and FTS reactions of CO₂.

### SUMMARY OF THE INVENTION

An object of the present invention is to provide a catalyst for hydrogenation of carbon dioxide.

Another object of the present invention is to provide a method of preparing the catalyst.

Still another object of the present invention is to provide a method of synthesizing a hydrocarbon compound having 5 or more carbon atoms using the catalyst.

The method of preparing a sodium-catalyzed iron-aluminum inorganic catalyst according to the one embodiment of the present invention is a method of preparing a catalyst for catalyzing a hydrogenation reaction of carbon dioxide, and the method includes: a first step of mixing a precipitant solution formed by dissolving a basic precipitant with a reaction solution formed by dissolving an iron (Fe) precursor material and an aluminum (Al) precursor material, thereby forming a suspension solution; a second step of aging the suspension solution; a third step of separating powder from the aged suspension solution; a fourth step of drying the separated powder and then performing a first heat treatment, thereby forming first catalyst powder; and a fifth step of adding and stirring the first catalyst powder and a sodium precursor material to water and evaporating the water and then performing a second heat treatment, thereby preparing a sodium-introduced iron-aluminum inorganic catalyst.

In the one embodiment, the iron (Fe) precursor material may include iron nitride, and the aluminum (Al) precursor material may include aluminum (Al) nitride.

In the one embodiment, the iron precursor and the aluminum precursor may be mixed at a molar ratio of 1:0.9 to 1:1.1 in the reaction solution.

In the one embodiment, the basic precipitant may include sodium carbonate (Na₂CO₃) or ammonium carbonate ((NH₄)₂CO₃).

In the one embodiment, the basic precipitant solution may be added such that pH of the reaction solution is about 6.5 to about 9.0.

In the one embodiment, the first heat treatment may be performed for 5 hours to 7 hours at a temperature of 400° C. to 650° C. and under an air flow condition, and first catalyst powder containing iron oxide, aluminum oxide, iron-aluminum oxide or the like may be formed by the first heat treatment.

In the one embodiment, the sodium precursor may include sodium carbonate.

In the one embodiment, the second heat treatment may be performed for 5 hours to 7 hours at a temperature of 400° C. to 650° C. and under an air flow condition.

The sodium-catalyzed iron-aluminum inorganic catalyst according to the one embodiment of the present invention refers to a catalyst for catalyzing the hydrogenation reaction of carbon dioxide, and may have a composition in which sodium is introduced on surfaces of iron-aluminum oxide particles or inside thereof to have a concentration of 3 wt % to 30 wt %.

In the one embodiment, the sodium may have the concentration of 8 wt % to 20 wt %.

A method of synthesizing an alpha olefin according to the one embodiment of the present invention includes: a first step of reducing a sodium-catalyzed iron-aluminum composite catalyst in a tubular reactor; and a second step of supplying mixed gas of hydrogen and carbon dioxide into the tubular reactor to induce a hydrogenation reaction of carbon dioxide, thereby producing an alpha olefin having 5 or more carbon atoms.

The sodium-catalyzed iron-aluminum inorganic catalyst according to the present invention can form long-chain hydrocarbons of C5+ with high selectivity under various conditions while ensuring long-term catalyst safety in which Fe for providing Fe₅C₂ as a main active site, Al₂O₃ for helping the stabilization and reaction of active sites, and Na for catalyzing the reaction systematically participate in the hydrogenation reaction of carbon dioxide.

### BRIEF DESCRIPTION OF THE DRAWINGS

FIG. 1 is a flowchart for illustrating a method of preparing a sodium-catalyzed iron-aluminum inorganic catalyst according to the one embodiment of the present invention.

FIG. 2 is a view for illustrating a reaction mechanism of the sodium-catalyzed iron-aluminum inorganic catalyst for a carbon dioxide hydrogenation reaction.

FIG. 3 is a graph showing performance evaluation results in the carbon dioxide hydrogenation reaction of an FeAlOₓ—Na(y) catalyst according to changes in Na concentration (y).

FIG. 4 is a graph showing performance evaluation results in the carbon dioxide hydrogenation reaction of an FeAlOₓ—Na(20) catalyst having an Na concentration of 20 wt % according to changes in spatial velocity of synthesis gas.

FIG. 5 is a graph measuring a hydrocarbon distribution of a product of the carbon dioxide hydrogenation reaction performed using the FeAlOₓ—Na(20) catalyst having an Na concentration of 20 wt %.

FIG. 6 is a graph showing results of measuring a long-term conversion rate of the FeAlOₓ—Na(20) catalyst with respect to the carbon dioxide hydrogenation reaction performed under a GHSV condition of 4000 mL g⁻¹ h⁻¹ (CO₂=1000 mL g⁻¹ h⁻¹, H₂=3000 mL g⁻¹ h⁻¹).

FIG. 7 is a graph showing results of measuring the long-term conversion rate of the FeAlOₓ—Na(20) catalyst with respect to the carbon dioxide hydrogenation reaction performed under a GHSV condition of 12000 mL g⁻¹ h⁻¹ (CO₂=3000 mL g⁻¹ h⁻¹, H₂=9000 mL g⁻¹ h⁻¹).

FIG. 8 shows images of a Wavelet analysis of X-ray adsorption (XAS) in a reduced state, a consumed state and an inactivated state of the FeAlOₓ—Na(y) catalyst.

FIG. 9 shows a Mossbauer analysis image of a performance-decreased FeAlOₓ—Na(y) catalyst.

FIG. 10 shows high-resolution transmission electron microscope (HR-TEM) images (A-E), TEM/Energy dispersive spectrometry (EDS) images (F-L), and line EDS results (M,N) of a part marked in image L of the FeAlOₓ—Na (20) catalyst.

FIG. 11 shows XRD images of the FeAlOₓ—Na(20) catalyst measured after reduction, after 75 hours of reaction (GHSV 4000 mL g⁻¹ h⁻¹), after 700 hours of reaction (GHSV 4000 mL g⁻¹ h⁻¹), and after 1350 hours of reaction (GHSV 12000 mL g⁻¹ h⁻¹).

### DETAILED DESCRIPTION OF THE INVENTION

Hereinafter, a catalyst for a hydrogenation reaction of carbon dioxide according to the one embodiment of the present invention and a method of preparing the same will be described in detail with reference to the accompanying drawings.

Since the present invention may be modified in various ways and have various forms, specific embodiments will be illustrated in the drawings and described in detail herein.

However, it will be understood that there is no intent to limit the present invention to the particular forms disclosed, but on the contrary, the present invention is to cover all modifications, equivalents, and alternatives falling within the spirit and scope of the present invention.

In describing each drawing, similar reference numerals are used for similar components.

In the accompanying drawings, the dimensions of structures are illustrated in an enlarged manner in order to clarify the present invention.

It will be understood that, although the terms first, second and the like may be used herein to describe various elements, these elements will not be limited by the above terms.

The terms are used only to distinguish one component from another component.

For example, a first element may be termed a second element, and, similarly, the second element may be termed the first element, without departing from the scope of the present invention.

The terms used herein are for the purpose of describing particular embodiments only and are not intended to limit the present invention.

Singular expressions include plural expressions unless the context clearly indicates otherwise.

In the present application, it will be understood that terms such as “include” or “have” are intended to designate the existence of features, numbers, steps, operations, components, parts, or combinations thereof described in the specification, and are not intended to preclude the possibility that one or more other features, numbers, steps, operations, components, parts, or combinations thereof may exist or may be added.

Unless otherwise defined, all terms used herein, including technical or scientific terms, have the same meanings as those generally understood by those skilled in the art to which the present invention pertains.

It will be further understood that terms, such as those defined in commonly used dictionaries, should be interpreted as having a meaning that is consistent with their meaning in the context of the relevant art and will not be interpreted in an idealized or overly formal sense unless expressly so defined herein.

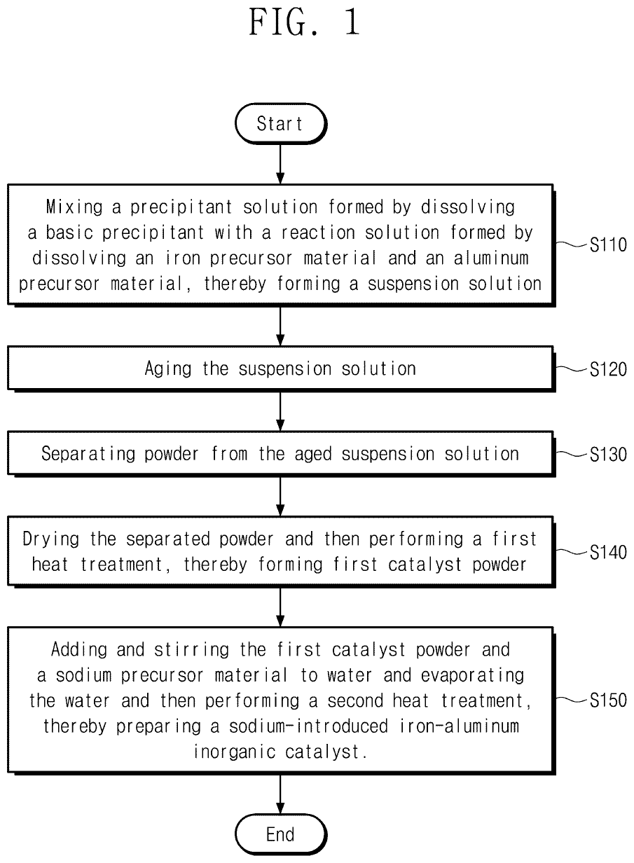

FIG. 1 is a flowchart for illustrating a method of preparing a sodium-catalyzed iron-aluminum inorganic catalyst according to the one embodiment of the present invention.

Referring to FIG. 1, the method of preparing a sodium-catalyzed iron-aluminum inorganic catalyst according to the one embodiment of the present invention includes: a first step S110 of mixing a precipitant solution formed by dissolving a basic precipitant with a reaction solution formed by dissolving an iron (Fe) precursor material and an aluminum (Al) precursor material, thereby forming a suspension solution; a second step S120 of aging the suspension solution; a third step S130 of separating powder from the aged suspension solution; a fourth step S140 of drying the separated powder and then performing a first heat treatment, thereby forming first catalyst powder; and a fifth step S150 of adding and stirring the first catalyst powder and a sodium precursor material to water and evaporating the water and then performing a second heat treatment, thereby preparing a sodium-introduced iron-aluminum inorganic catalyst.

The sodium-catalyzed iron-aluminum inorganic catalyst prepared according to the present invention may be used as a catalyst of a reaction for directly reacting carbon dioxide (CO₂) and hydrogen (H₂) to selectively produce a liquid hydrocarbon compound, for example, a long-chain hydrocarbon of C5+.

In the first step S110, the iron precursor material is not particularly limited as long as it is a material capable of providing iron (Fe) ions to the reaction solution, and may include, for example, iron nitride. The aluminum precursor material is not particularly limited as long as it is a material capable of providing aluminum (Al) ions to the reaction solution and may include, for example, aluminum (Al) nitride.

In the one embodiment, the solvent of the reaction solution is not particularly limited as long as it can dissolve the iron precursor and the aluminum precursor, and for example, water such as deionized water may be used as the solvent.

In the reaction solution, the iron precursor and the aluminum precursor may be mixed at the molar ratio of about 1:0.5 to about 1:1.5. In addition, the total concentration of the iron precursor and the aluminum precursor in the reaction solution may be about 0.1 mol/L to 1.0 mol/L.

In the one embodiment, the basic precipitant may precipitate a reaction product of iron ions dissociated from the iron precursor and aluminum ions dissociated from the aluminum precursor by adjusting the suspension solution so as to be basic. As the basic precipitant, a basic compound may be used without limitation. In the one embodiment, the basic precipitant may include sodium carbonate such as sodium carbonate (Na₂CO₃) or ammonium carbonate such as ammonium carbonate ((NH₄)₂CO₃).

The solvent of the basic precipitant solution may be the same as the solvent of the reaction solution, and the basic precipitant solution may be added such that the reaction solution has pH of about 6.5 to about 9.0. Meanwhile, in the basic precipitant solution, the sodium carbonate or the ammonium carbonate may have a concentration of about 1 mol/L to about 5 mol/L. For example, in the basic precipitant solution, the sodium carbonate or the ammonium carbonate may have the concentration of about 1.5 mol/L to about 3 mol/L.

In the one embodiment, in order to form the suspension solution, the precipitant solution may be added dropwise to the reaction solution and stirred at pH of about 6.5 to about 9 for about 12 hours to 15 hours under temperature conditions of about 60° C. to about 120° C. and stirring conditions of about 200 RPM to about 350 RPM. In the one embodiment, a brown precipitate may be formed by the dropwise addition of the precipitant solution to the reaction solution.

In the second step S120, the suspension solution may be aged while being maintained in a sealed container at about 20° C. to about 40° C. for about 4 hours to 24 hours.

In the third step S130, powder generated by the reaction of the iron ions and the aluminum ions may be filtered and separated from the aged suspension, and the separated powder may be washed using deionized water.

In the fourth step S140, the separated powder may be dried at a temperature of about 60° C. to about 100° C. for about 10 hours to about 14 hours, and then first heat treated for about 5 hours to about 7 hours at a temperature of about 400° C. to about 650° C. and under an air flow condition, and first catalyst powder containing iron oxide, aluminum oxide, iron-aluminum oxide, or the like may be formed by the first heat treatment.

In the fifth step S150, the first catalyst powder and the sodium precursor material may be added and stirred to water, the water is evaporated and then a second heat treatment is performed, thereby preparing an iron-aluminum inorganic catalyst into which sodium is introduced.

In the one embodiment, the sodium precursor may include sodium carbonate. The sodium precursor may be added such that the ratio of an introduced sodium mass to a total mass of the sodium-introduced iron-aluminum inorganic catalyst ([Na mass]/[total catalyst mass]) is about 5 wt % to about 30 wt %.

In the one embodiment, the second heat treatment may be performed for about 5 hours to about 7 hours at a temperature of about 400° C. to about 650° C. and under an air flow condition.

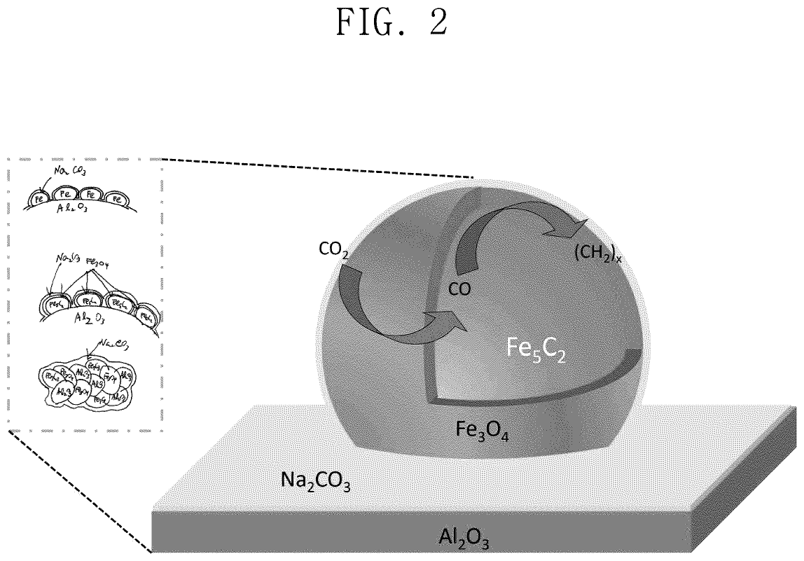

FIG. 2 is a view for illustrating a reaction mechanism of the sodium-catalyzed iron-aluminum inorganic catalyst for a carbon dioxide hydrogenation reaction.

Referring to FIG. 2, the sodium-catalyzed iron-aluminum inorganic catalyst may form long-chain hydrocarbons of C5+ with high selectivity under various conditions in which Fe for providing Fe₅C₂ as a main active site, Al₂O₃ for helping the stabilization and reaction of active sites, and Na for catalyzing the reaction systematically participate in the hydrogenation reaction of carbon dioxide.

In the one embodiment, in order to improve the conversion rate of carbon dioxide and the selectivity of the long-chain hydrocarbons of C5+, the sodium-catalyzed iron-aluminum inorganic catalyst may have a composition in which sodium is introduced on surfaces of iron-aluminum oxide particles or inside thereof to have a concentration of about 5 wt % to about 30 wt % ([Na mass]/[total catalyst mass]). When the concentration of sodium is less than 5 wt % or greater than 30 wt %, the conversion rate of carbon dioxide and the selectivity of long-chain hydrocarbon of C5+ of the product may be deteriorated in the hydrogenation reaction of carbon dioxide.

In the one embodiment of the present invention, the method for synthesizing liquid hydrocarbon through a hydrogenation reaction of carbon dioxide using the sodium-catalyzed iron-aluminum inorganic catalyst may include: a first step of reducing the sodium-catalyzed iron-aluminum inorganic catalyst in a tubular reactor; and a second step of supplying mixed gas of hydrogen and carbon dioxide into the tubular reactor to induce a hydrogenation reaction of carbon dioxide, thereby producing the long-chain hydrocarbon of C5+.

In the first step, hydrogen gas flows into a tubular reactor to which the sodium-catalyzed iron-aluminum inorganic catalyst is fixed, so that at least a portion of an iron oxide phase of the catalyst may be reduced into metal iron. In particular, when the catalyst includes sodium, the reduction of the iron oxide may be catalyzed to form more metal iron, and the above metal iron may form an iron carbide phase (Fe₅C₂, Fe₇C₃) acting as an active point in a reaction for forming long-chain hydrocarbon of C5+ by carbon species generated due to decomposition of carbon dioxide.

In the one embodiment, in order to reduce the sodium-catalyzed iron-aluminum inorganic catalyst, the sodium-catalyzed iron-aluminum inorganic catalyst may be fixed in the tubular reactor and then the hydrogen gas may flow for about 4 hours to about 8 hours while raising a temperature to about 320° C. to about 450° C. at a temperature increase rate of about 1° C. to about 5° C. At this time, a pressure inside the tubular reactor may be adjusted to about 3.5 MPa to about 5.0 MPa. Meanwhile, The sodium-catalyzed iron-aluminum inorganic catalyst may be mixed with silica powder, which is a thermal diluent, and then fixed inside the tubular reactor by a porous support such as quartz wool.

In the second step, the temperature inside the tubular reactor to which the reduced sodium-catalyzed iron-aluminum inorganic catalyst is fixed may be adjusted to about 270° C. to about 370° C., and then mixed gas (H₂/CO₂) of hydrogen (H₂) and carbon dioxide (CO₂) may flow, so that the long-chain hydrocarbon of C5+ may be produced. When the temperature inside the tubular reactor is less than 270° C., the yield of the long-chain hydrocarbon of C5+ may be lowered due to insufficient activation of carbon dioxide. When the temperature inside the tubular reactor exceeds 370° C., the catalyst may structurally collapse, and the RWGS reaction of carbon dioxide occurs more predominantly due to re-oxidation of a metal Fe phase caused by agglomeration of nanoparticles. For example, the temperature inside the tubular reactor to which the catalyst is fixed may be adjusted to about 320° C. to about 360° C.

In the one embodiment, for the hydrogenation reaction of carbon dioxide, the pressure inside the tubular reactor may be adjusted to about 3.5 MPs or more, and then the mixed gas (H₂/CO₂) may be supplied into the tubular reactor at a predetermined speed. When the pressure inside the reactor is less than 3.5 MPa, re-adsorption of hydrocarbons of C2 to C4 may be decreased, and thus the yield of hydrocarbon of C5+ may be deteriorated. For example, the pressure inside the tubular reactor may be adjusted to about 3.5 MPa to about 5.0 MPa.

In the one embodiment, for the hydrogenation reaction of carbon dioxide, gas formed by mixing hydrogen (H₂) with carbon dioxide (CO₂) at the ratio of about 2.5:1 to about 3.5:1 may be supplied into the tubular reactor. When the H₂/CO₂ ratio is less than 2.5, the FTS reaction may be deteriorated due to insufficient hydrogen. When the ratio exceeds 3.5, the FTS reaction may be deteriorated due to re-oxidation of the metal Fe phase of the catalyst by agglomeration of particles. Meanwhile, The mixed gas may be supplied to the tubular reactor at GHSV of about 4000 to 20000 mL g⁻¹ h⁻¹.

Hereinafter, specific embodiments of the present invention will be described in detail. However, the following embodiments are merely some exemplary embodiments of the present invention, and the scope of the present invention is not limited to the following embodiments.

### EMBODIMENTS

A sodium-catalyzed iron-aluminum inorganic catalyst (FeAlOₓ—Na(y), where y represents a concentration of Na) was synthesized using a co-precipitation scheme, and a hydrogenation reaction of carbon dioxide was performed using the synthesized catalyst.

First, 15 g of iron nitrate and 15 g of aluminum nitrate were distilled and dissolved in 228 g of distilled and deionized (DDI) water to prepare a reaction solution, and 15 g of ammonium carbonate or sodium carbonate was dissolved in 78 g of DDI water to prepare a precipitant solution.

Subsequently, while stirring the reaction solution heated to 60° C. to 115° C. at 200 RPM to 350 RPM, the precipitant solution was added dropwise thereto by using a dropper until pH reached 6.8 to 9.

Subsequently, the reaction solution to which the precipitant solution was added dropwise was stirred for 13 hours in a sealed bottle and then aged at room temperature for 6 hours without stirring. At this time, a brown precipitate was formed.

Subsequently, the aged suspension was filtered and then washed by DDI water. The collected powder was dried at 60° C. to 100° C. for at least 12 hours, and the dried powder was calcined at 600° C. for at least 6 hours under air flow conditions (flow rate 100 mL h⁻¹).

Subsequently, the calcined powder was put into water together with sodium carbonate and then stirred. The water was evaporated and then the powder without the water was calcined at 600° C. for 6 hours or more under air flow conditions (flow rate 100 mL h⁻¹), thereby preparing a sodium-catalyzed iron-aluminum inorganic catalyst.

### [Experimental Example 1]: Hydrogenation Performance

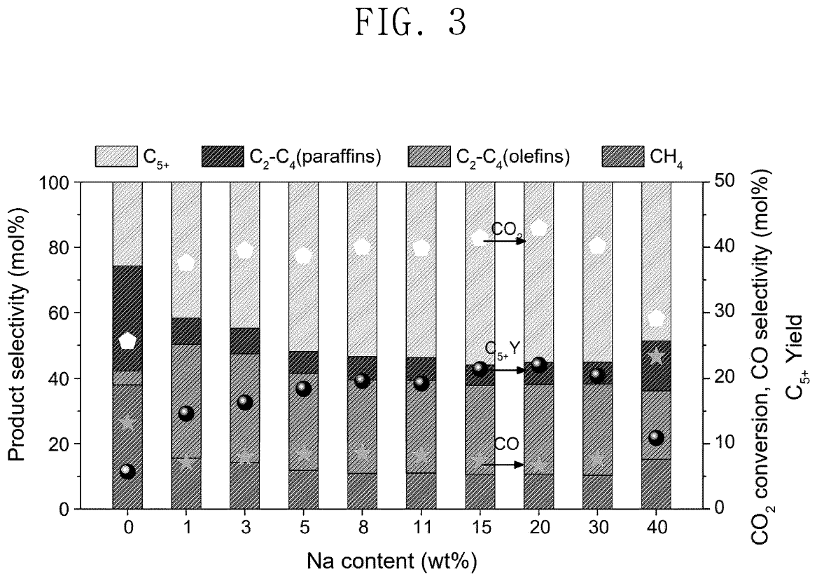

FIG. 3 is a graph showing performance evaluation results in the carbon dioxide hydrogenation reaction of an FeAlOₓ—Na(y) catalyst according to changes in Na concentration (y). Table 1 below shows the CO₂ conversion rate, the selectivity of long-chain hydrocarbon of C5+, and the yield of long-chain hydrocarbon of C5+ including CO in a product, which are analyzed from FIG. 3.

The hydrogenation reaction of carbon dioxide was performed by allowing synthesis gas having the H₂/CO₂ ratio of 3 into a tubular reactor heated to 330° C. and having an FeAlOₓ—Na(y) catalyst fixed therein to flow at a spatial velocity of 4000 mL g⁻¹ h⁻¹ as shown in Table 1.

TABLE 1

| | | | GHSV | | | C₅₊ |
|---|---|---|---|---|---|---|
| | Reaction | H₂/ | (mL | | | Yield |
| y | temperature | CO₂ | g⁻¹ | Conversion | C₅₊ | with |
| (wt %) | (° C.) | ratio | h⁻¹) | rate | Selectivity | CO |
| 0 | 330 | 3 | 4000 | 25.6 | 22.2 | 5.7 |
| 1 | | | | 37.9 | 39.3 | 14.9 |
| 3 | | | | 39.7 | 41.8 | 16.6 |
| 5 | | | | 41.1 | 52.8 | 21.7 |
| 8 | | | | 43.2 | 56.7 | 24.5 |
| 11 | | | | 43.3 | 56.1 | 24.3 |
| 15 | | | | 41.7 | 52.5 | 21.9 |
| 20 | | | | 43.5 | 52.4 | 22.8 |
| 30 | | | | 40.2 | 50.7 | 20.4 |
| 40 | | | | 29.1 | 37.4 | 10.9 |

Referring to FIG. 3 and Table 1, when the concentration of Na is 5 wt % or more in the FeAlOₓ—Na(Y) catalyst, the high CO₂ conversion rate greater than 40% and the selectivity of long-chain hydrocarbon of C5+ greater than 20% were achieved. In particular, when the concentration of Na is about 8 wt % to about 20 wt %, the CO₂ conversion and the selectivity of long-chain hydrocarbon of C5+ were expressed higher.

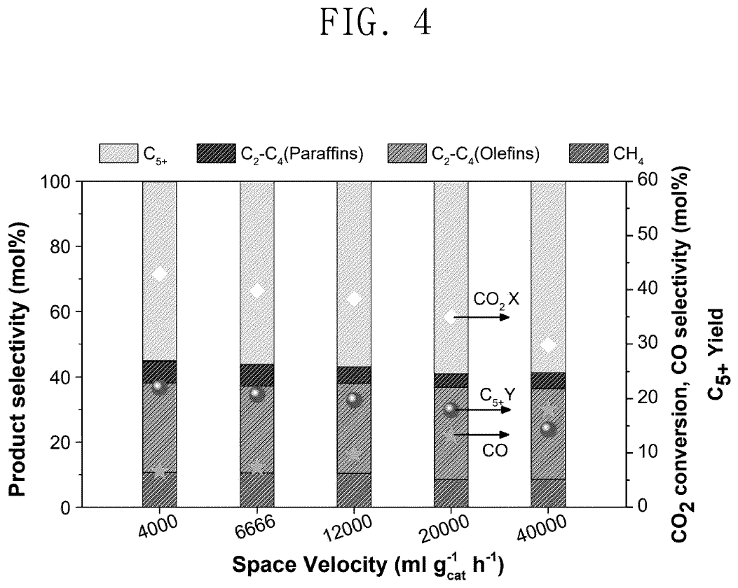

FIG. 4 is a graph showing performance evaluation results in the carbon dioxide hydrogenation reaction of an FeAlOₓ—Na(20) catalyst having an Na concentration of 20 wt % according to changes in GHSV of the mixed gas of CO₂ and H₂. Table 2 below shows the CO₂ conversion and the selectivity of long-chain hydrocarbon of C5+, which are analyzed from FIG. 4.

TABLE 2

| | | | GHSV | | | C₅₊ |
|---|---|---|---|---|---|---|
| | Reaction | H₂/ | (mL | | | Yield |
| y | temperature | CO₂ | g⁻¹ | Conversion | C₅₊ | with |
| (wt %) | (° C.) | ratio | h⁻¹) | rate | Selectivity | CO |
| 20 | 330 | 3 | 4000 | 42.9 | 51.28 | 22 |
| | | | 6666 | 39.8 | 52.01 | 20.7 |
| | | | 12000 | 38.3 | 51.43 | 19.7 |
| | | | 2000 | 35 | 51.14 | 17.9 |
| | | | 4000 | 29.8 | 47.98 | 14.3 |

Referring to FIG. 4 and Table 2, it was shown that the CO₂ conversion and the selectivity of long-chain hydrocarbon of C5+ were decreased when the GHSV of the mixture gas of CO₂ and H₂ supplied into the tubular reactor is 20000 mL g⁻¹ h⁻¹ or more.

Table 3 shows performance evaluation results in the carbon dioxide hydrogenation reaction of the FeAlOₓ—Na(20) catalyst having an Na concentration of 20 wt % according to changes in reaction temperature.

TABLE 3

| | | | GHSV | | | C₅₊ |
|---|---|---|---|---|---|---|
| | Reaction | H₂/ | (mL | | | Yield |
| y | temperature | CO₂ | g⁻¹ | Conversion | C₅₊ | with |
| (wt %) | (° C.) | ratio | h⁻¹) | rate | Selectivity | CO |
| 20 | 330 | 1.5 | 5000 | 26.4 | 58.33 | 15.4 |
| | 370 | | | 30 | 57.67 | 17.3 |

Referring to Table 3, the higher CO₂ conversion and the higher selectivity of long-chain hydrocarbon of C5+ were achieved when the reaction temperature inside the tubular reactor was 370° C. higher than 330° C.

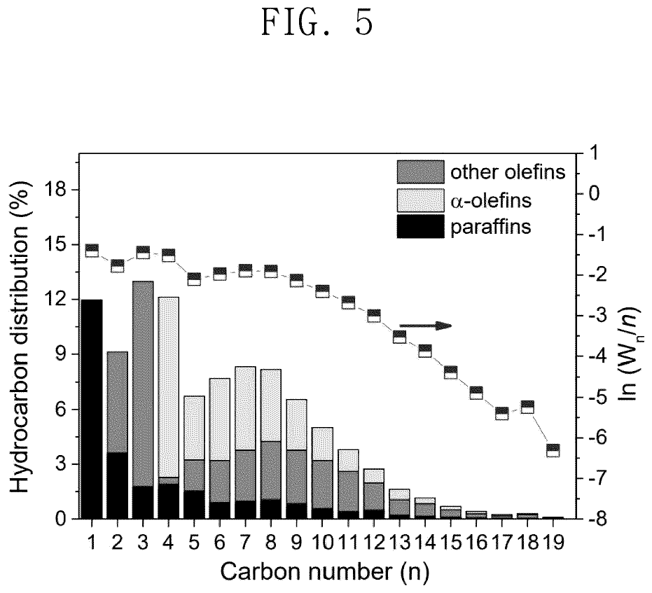

FIG. 5 is a graph measuring a hydrocarbon distribution of a product of the carbon dioxide hydrogenation reaction performed using the FeAlOₓ—Na(20) catalyst having an Na concentration of 20 wt %. At this time, the hydrogenation reaction of carbon dioxide was performed under conditions of a reaction temperature of 330° C., a reaction pressure of 3.5 MPa, H₂/CO₂ of 3:1, and GHSV of 4000 mL g⁻¹ h⁻¹ (CO₂=1000 mL g⁻¹ h⁻¹, H₂=3000 mL g⁻¹ h⁻¹).

Referring to FIG. 5, it can be confirmed that the FeAlOₓ—Na(20) catalyst exhibits excellent selectivity of long-chain hydrocarbon of C5+.

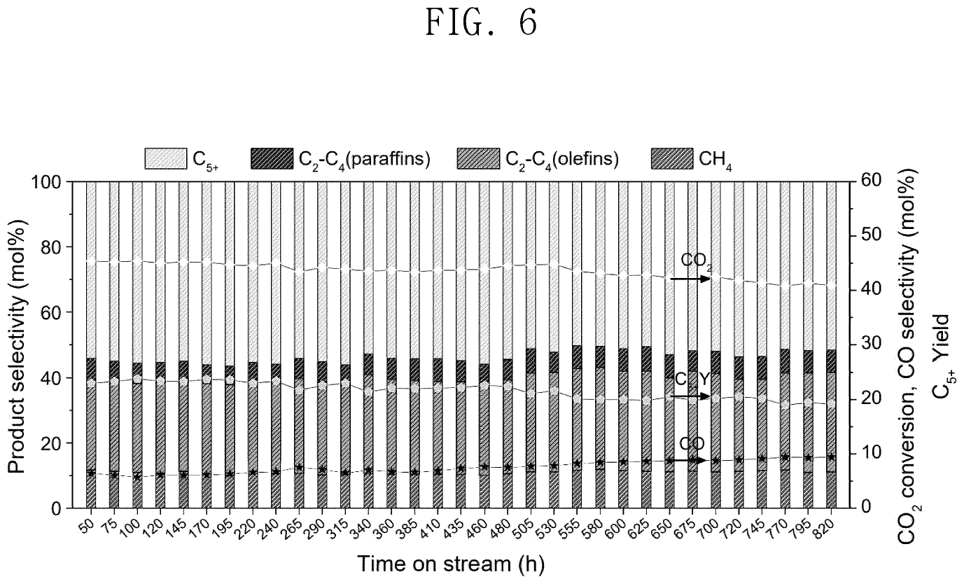

FIG. 6 is a graph showing results of measuring a long-term operability of the FeAlOₓ—Na(20) catalyst with respect to the carbon dioxide hydrogenation reaction performed under a GHSV condition of 4000 mL g⁻¹ h⁻¹ (CO₂=1000 mL g⁻¹ h⁻¹, H₂=3000 mL g⁻¹ h⁻¹). The hydrogenation reaction of carbon dioxide was performed under conditions of a reaction temperature of 330° C., a reaction pressure of 3.5 MPa, and H₂/CO₂ of 3:1.

Referring to FIG. 6, it was found that the performance of the FeAlOₓ—Na(20) catalyst was stably maintained for 700 hours.

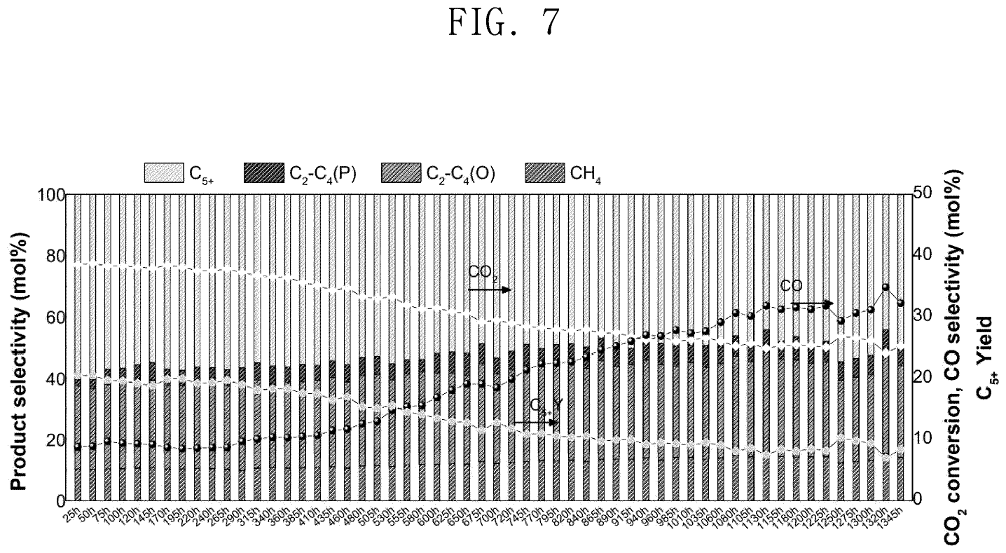

FIG. 7 is a graph showing results of measuring the long-term conversion rate of the FeAlOₓ—Na(20) catalyst with respect to the carbon dioxide hydrogenation reaction performed under a GHSV condition of 12000 mL g⁻¹ h⁻¹ (CO₂=3000 mL g⁻¹ h⁻¹, H₂=9000 mL g⁻¹ h⁻¹). The hydrogenation reaction of carbon dioxide was performed under conditions of a reaction temperature of 330° C., a reaction pressure of 3.5 MPa, and H₂/CO₂ of 3:1.

Referring to FIG. 7, it was found that the FeAlOₓ—Na(20) catalyst had activity for at least 1350 hours even under high GHSV conditions.

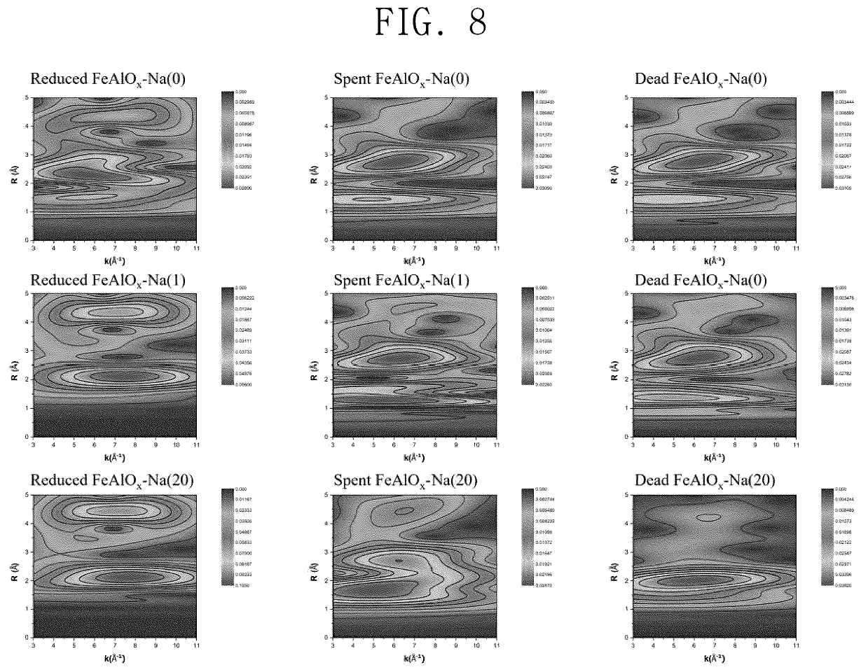

FIG. 8 shows images of a Wavelet analysis of X-ray adsorption (XAS) in a reduced state, a consumed state and an inactivated state of the FeAlOₓ—Na(y) catalyst.

Referring to FIG. 8, it was shown that the reduction of the catalyst was promoted due to the introduction of Na, and thus the iron state was reduced to metal iron other than iron oxide. In addition, it was confirmed that, a high proportion of iron carbide phase (Fe₅C₂, Fe₇C₃) was present in the case of a catalyst having high reactivity.

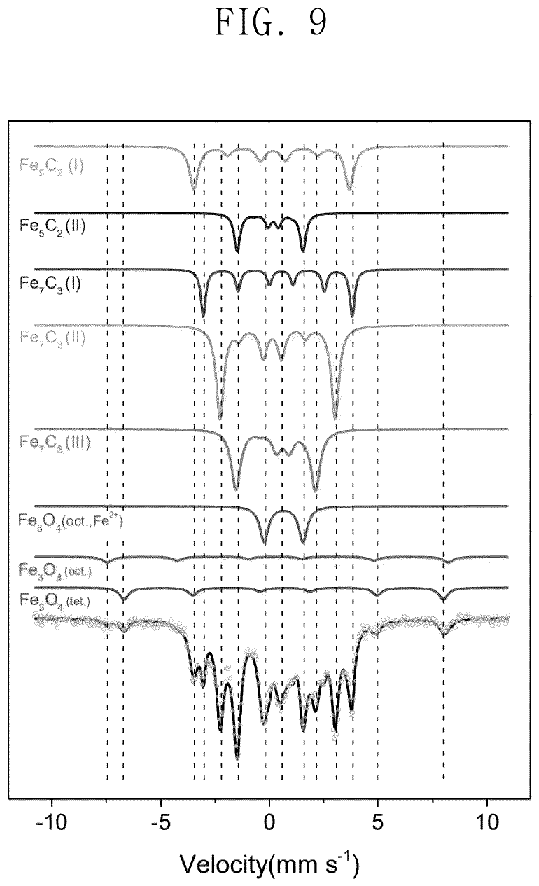

FIG. 9 shows a Mossbauer analysis image of a performance-decreased FeAlOₓ—Na(y) catalyst.

Referring to FIG. 9, it can be confirmed that a deactivation pathway of an Na-doped FeAlOₓ—Na(y) catalyst had an active point Fe₅C₂ changed to Fe₇C₃ other than an active point Fe₅C₂ changed to Fe₂O₃ as in deactivation pathways of generally known prior catalysts. Since Fe₇C₃ has a lower activity compared to Fe₅C₂ but is not an inactive point like Fe₂O₃, FeAlOₓ—Na(y) may have activity for a relatively longer time than conventional iron-aluminum catalysts.

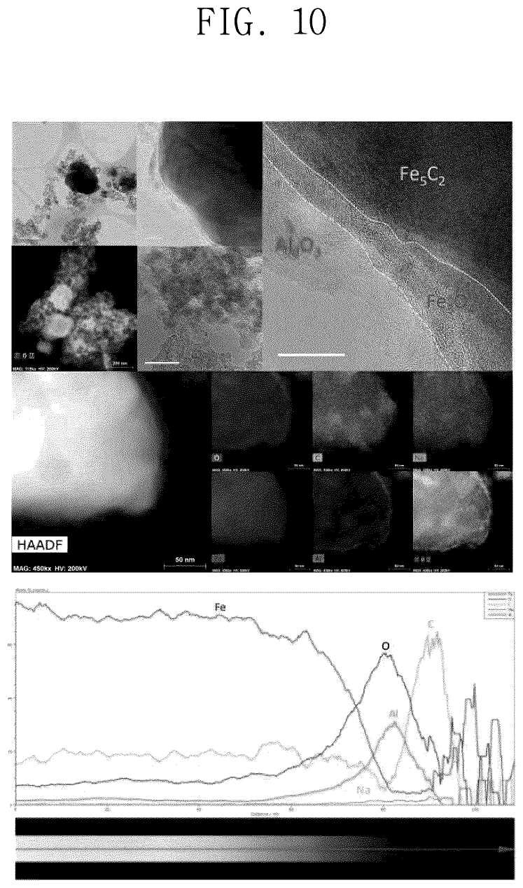

FIG. 10 shows HR-TEM images (A-E), TEM/EDS images (F-L), and line EDS results (M,N) of a part marked in image L of the FeAlOₓ—Na (20) catalyst.

Referring to FIG. 10, as confirmed in FIGS. 8 and 9, it can be seen that Fe₅C₂ as an active point of the FTS reaction and Fe₃O₄ as an active point of the RWGS reaction are properly harmonized in the FeAlOₓ—Na(20) catalyst doped with 20 wt % of Na, so that CO₂ as a reactant is effectively converted. In addition, it can be confirmed based on the Line EDS analysis results of TEM that Al components are uniformly coated on the surface of iron. It can be seen that Al serves to prevent Fe particles from agglomerating with each other during the reaction in the FeAlOₓ—Na(20) catalyst, thereby preventing the inactivity of the catalyst.

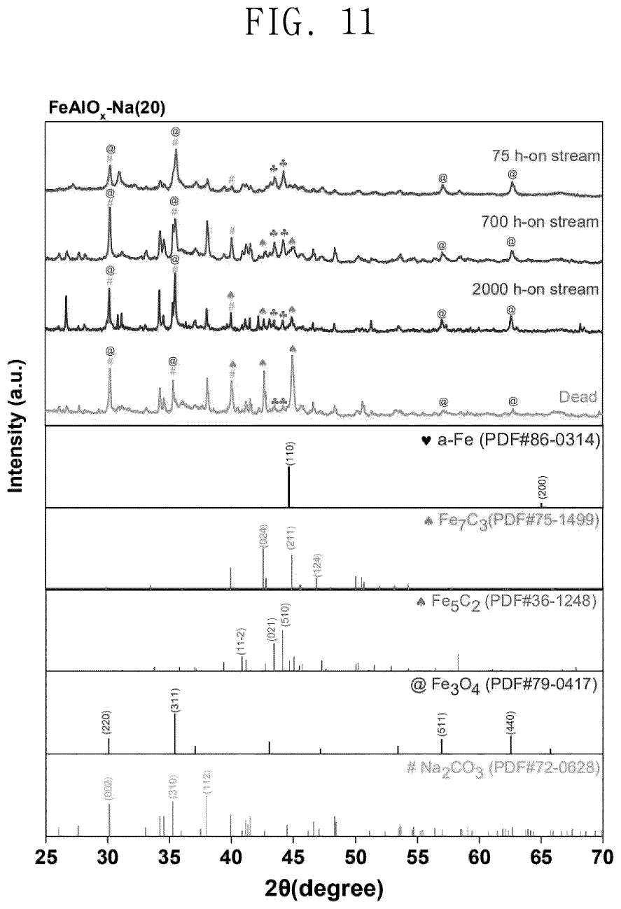

FIG. 11 shows XRD images of the FeAlOₓ—Na(20) catalyst measured after reduction, after 75 hours of reaction (GHSV 4000 mL g⁻¹ h⁻¹), after 700 hours of reaction (GHSV 4000 mL g⁻¹ h⁻¹), and after 1350 hours of reaction (GHSV 12000 mL g⁻¹ h⁻¹).

Referring to FIG. 11, it can be confirmed that, as the reaction proceeds, Fe₅C₂ is not converted into Fe₃O₄ or other oxides, but converted into Fe₇C₃ by carbonization. In addition, it can be confirmed by checking an XRD peak FWHM that the crystal size of the Fe₃O₄ decreases and the crystal of the Fe₇C₃ grows as the reaction proceeds. Stable Fe₇C₃ is also one of the active points of the FTS reaction so as to check whether the long-term operability of the FeAlOₓ—Na(20) catalyst can be ensured.

Although the above description has been made with reference to exemplary embodiments of the present invention, it will be understood by those skilled in the art that the present invention may be variously modified and changed without departing from the spirit and scope of the present invention described in the following claims.

What is claimed is:

1. A method of preparing a sodium-catalyzed iron-aluminum inorganic catalyst in a method of preparing a catalyst for catalyzing a hydrogenation reaction of carbon dioxide, and the method comprises:
a first step of mixing a precipitant solution formed by dissolving a basic precipitant with a reaction solution formed by dissolving an iron (Fe) precursor material and an aluminum (Al) precursor material, thereby forming a suspension solution;
a second step of aging the suspension solution;
a third step of separating powder from the aged suspension solution;
a fourth step of drying the separated powder and then performing a first heat treatment, thereby forming first catalyst powder; and
a fifth step of adding and stirring the first catalyst powder and a sodium precursor material to water and evaporating the water and then performing a second heat treatment, thereby preparing a sodium-introduced iron-aluminum inorganic catalyst.

2. The method of claim 1, wherein the iron (Fe) precursor material includes iron nitride, and the aluminum (Al) precursor material includes aluminum (Al) nitride.

3. The method of claim 2, wherein the iron precursor and the aluminum precursor are mixed at a molar ratio of 1:0.5 to 1:1.5 in the reaction solution.

4. The method of claim 1, wherein the basic precipitant includes sodium carbonate (Na₂CO₃) or ammonium carbonate ((NH₄)₂CO₃).

5. The method of claim 4, wherein the basic precipitant solution is added such that the reaction solution has pH of about 6.5 to about 9.0.

6. The method of claim 1, wherein the first heat treatment is performed for 5 hours to 7 hours at a temperature of 400° C. to 650° C. and under an air flow condition, and first catalyst powder containing iron oxide, aluminum oxide, and iron-aluminum oxide is formed by the first heat treatment.

7. The method of claim 1, wherein the sodium precursor includes sodium carbonate.

8. The method of claim 7, wherein the second heat treatment is performed for 5 hours to 7 hours at a temperature of 400° C. to 650° C. and under an air flow condition.

9. A sodium-catalyzed iron-aluminum inorganic catalyst in a catalyst for catalyzing a hydrogenation reaction of carbon dioxide, the sodium-catalyzed iron-aluminum inorganic catalyst having a composition in which sodium is introduced on surfaces of iron-aluminum oxide particles or inside thereof to have a concentration of 3 wt % to 30 wt %.

10. The sodium-catalyzed iron-aluminum inorganic catalyst of claim 9, wherein the sodium has a concentration of 8 wt % to 20 wt %.

11. A method of synthesizing a long-chain hydrocarbons, the method comprising:
a first step of reducing a sodium-catalyzed iron-aluminum composite catalyst based on claim 8 in a tubular reactor; and
a second step of supplying mixed gas of hydrogen and carbon dioxide into the tubular reactor to induce a hydrogenation reaction of carbon dioxide, thereby generating long-chain hydrocarbon having 5 or more carbon atoms.
